(19) United States
(12) Patent Application Publication
(10) Pub. No.: US 2024/0316539 A1
(43) Pub. Date: Sep. 26, 2024

(54) POROUS CATALYST AND METHOD OF USE FOR THE TANDEM CAPTURE AND CONVERSION OF CARBON DIOXIDE TO HYDROCARBONS

(71) Applicant: UT-Battelle, LLC, Oak Ridge, TN (US)

(72) Inventor: Michelle K. Kidder, Clinton, TN (US)

(21) Appl. No.: 18/611,817

(22) Filed: Mar. 21, 2024

Related U.S. Application Data
(60) Provisional application No. 63/453,768, filed on Mar. 22, 2023.

(51) Int. Cl. B01J 23/889; B01J 23/00; B01J 23/04; B01J 35/45; B01J 35/53; B01J 35/64; C07C 1/12

(52) U.S. Cl. CPC B01J 23/8892; B01J 23/005; B01J 23/04; B01J 35/45; B01J 35/53; B01J 35/643; B01J 35/647; C07C 1/12; C07C 2523/72; C07C 2523/745; C07C 2523/75; C07C 2523/755; C07C 2523/80; C07C 2523/83; C07C 2523/847; C07C 2523/889

(57) ABSTRACT

A porous catalyst useful in the conversion of carbon dioxide to one or more hydrocarbons, the porous catalyst containing: (i) a bimetallic oxide portion containing at least one of iron oxide and nickel oxide or carbide in combination with at least one oxide, hydroxide, and/or carbide of at least one of manganese, cobalt, copper, yttrium, zirconium, niobium, hafnium, zinc, and lanthanides; and (ii) an alkali metal oxide, hydroxide, or carbonate portion in contact with the bimetallic oxide portion; wherein the porous catalyst contains pores in the bimetallic oxide portion. A method of using the porous catalyst to convert carbon dioxide to hydrocarbons, particularly olefins, containing at least four carbon atoms, is also described.

### CROSS REFERENCE TO RELATED APPLICATION

The present application claims benefit of U.S. Provisional Application No. 63/453,768, filed on Mar. 22, 2023, all of the contents of which are incorporated herein by reference.

### STATEMENT REGARDING FEDERALLY SPONSORED RESEARCH

This invention was made with government support under Prime Contract No. DE-AC05-00OR22725 awarded by the U.S. Department of Energy. The government has certain rights in the invention.

### FIELD OF THE INVENTION

This invention generally relates to catalysts and methods of use for the conversion of carbon dioxide into hydrocarbon products. The invention more particularly relates to catalytic methods for converting carbon dioxide to hydrocarbons, particularly olefins, particularly those hydrocarbons containing at least four carbon atoms.

### BACKGROUND

Hydrocarbons containing at least four carbon atoms are most commonly produced by the extraction and refining of fossil fuels, which is a non-renewable energy source. As well known, the extraction and refining of fossil fuels is energy intensive. The selective production of alpha-olefins containing at least four carbon atoms is particularly difficult, and primarily produced by the oligomerization of ethylene, which is in turn produced from fossil fuels.

In view of the steep energy expenditures associated with fossil fuel production of hydrocarbons, alternative means for hydrocarbon production continue to be investigated. There has been a particular interest in using carbon dioxide (CO₂) as a source for the production of hydrocarbons, particularly as carbon dioxide is a growing atmospheric waste product contributing to climate change. Such methods would have the potential to significantly reduce reliance on fossil fuels and reduce carbon dioxide emissions, which is an important step towards a carbon-neutral future. However, efforts in the conversion of carbon dioxide have been largely hindered by low production capacity and difficulty in producing larger chain hydrocarbons (typically those containing at least 4, 5, or 6 carbon atoms). Production of longer-chain alpha-olefins, in particular, from carbon dioxide would be particularly advantageous considering the importance of alpha-olefins in producing a wide range of commodities, such as plastics, polymers (e.g., polyolefins), lubricants, and detergents.

### SUMMARY

In one aspect, the present disclosure is directed to porous catalysts useful in the conversion of carbon dioxide to one or more hydrocarbons. In some embodiments, the present disclosure provides porous microspheres that are high-surface, bimetallic oxide catalysts comprising carbon capture basic sites for the synchronous capture and activation of CO₂ to C₄₋₁₅ olefins (i.e., C₄₋₁₅⁼, or more particularly, α-C₄₋₁₅⁼) via tandem reactions. In some embodiments, the technologies described herein provide a porous bimetallic oxide, such as at least one oxide or carbide form of Fe, Co, Cu, Mn, Zr, Zn, decorated with an alkali metal oxide, hydroxide, or carbonate form. The porous bimetallic oxide acts as a microreactor for paraffin and olefin production, while the alkali metal oxide, hydroxide, or carbonate acts as a carbon dioxide capture site followed by the chemical transformation and transport to the porous bimetallic oxide. In some embodiments, the catalyst microspheres have a core-shell arrangement in which the core contains the bimetallic oxide or carbide portion and the shell contains the alkali metal oxide, hydroxide, or carbonate portion. In other embodiments, the catalyst may be coated onto a substrate. The catalyst on the substrate may, in some embodiments, be the microspheres described above. In other embodiments, the catalyst coated onto a substrate may be composed of, for example, a first layer containing the bimetallic oxide portion and a second layer containing the alkali metal oxide, hydroxide, or carbonate portion, wherein the second layer is in contact with the first layer, and the second layer is uncoated and in free contact with its gaseous environment. In either case, the coating thickness may be appropriately selected to improve the selectively of C₄⁺ hydrocarbon products by optimizing the residence time along the surface and in the bulk.

More particularly, the porous catalysts contain at least (or exclusively) the following components: (i) a bimetallic oxide portion comprising at least one of iron oxide and nickel oxide in combination with at least one oxide, hydroxide, and/or carbide of at least one of manganese, cobalt, copper, yttrium, zirconium, niobium, hafnium, zinc, and lanthanides; and (ii) an alkali metal oxide, hydroxide, or carbonate portion in contact with the bimetallic oxide portion, wherein the porous catalyst contains pores in the bimetallic oxide portion. Component (ii) functions to absorb the carbon dioxide and transport it into component (i), wherein component (i) is the reactive portion that converts the carbon dioxide to hydrocarbons under appropriate conditions of temperature and pressure, as further described below. The catalysts described herein can advantageously convert carbon dioxide to hydrocarbons, particularly alpha-olefins, containing at least four, five, or six carbon atoms, preferably with a selectivity of at least 50 vol %.

In other embodiments, the catalyst materials can be 3D printed for better reactor designs that can overcome pressure drop issues and improved product distribution. In further embodiments, electrospinning can help metal distribution, improving product selectivity. The materials can advantageously be scaled into reactor beds quickly by manufactured design processes (e.g., support structures from 3D printing, monoliths, or electrospinning polymetallic fibers) which are advantageous over pellet and packed bed design and will aid chemical transformation, diffusion, mass transport, heat transfer and overcome pressure drop issues.

The hierarchical porous catalysts described herein can be coated onto monoliths, 3D printed into microchannels, or electrospun into polymetallic fibers. For example, the metal oxides (similar to ceramic materials) can be printed or coated onto surfaces into ideal geometric configurations and thicknesses. Printing provides the advantage of fitting the catalyst into different or existing reactor beds, where the channels that gas flow can be tuned to a specific size or “tortuosity” to enhance the proper selective product formation through enhanced sorption on surfaces. Coating of monoliths will be potentially advantageous as well for overcoming pressure drop issues in reactor beds.

In another aspect, the present disclosure is directed to a method for converting carbon dioxide to one or more hydrocarbons containing at least four carbon atoms. The hydrocarbons are typically composed of only carbon and hydrogen, but may or may not include an oxygen atom, which may result in an ether, alcohol, or ketone (e.g., dimethyl ether, methanol, or acetone). The method achieves this by contacting carbon dioxide gas and hydrogen gas with a porous catalyst, as described above, at a temperature of 100-800° C. and a pressure of 1-20 atm to result in conversion of the carbon dioxide to the one or more hydrocarbons. The method advantageously converts carbon dioxide to hydrocarbon product, with high selectivity and low energy input. In some embodiments, the method achieves production of at least 20, 30, 40, 50, 60, 70, 80, or 90 vol % of hydrocarbons (e.g., alkanes and/or olefins) containing at least four carbon atoms. The porous catalyst used in the method is also advantageously highly active, coke resistant, and stable. In separate or further embodiments, the porous catalyst is contained in a packed-bed reactor.

### BRIEF DESCRIPTION OF THE FIGURES

FIG. 1. A depiction of a printed monolith containing a porous bimetallic particle adorned with alkali metal cations.

FIG. 2. Graph showing the pore size distribution of porous FeMn@Na particles, as determined by nitrogen physisorption isotherm, wherein FeMn@Na corresponds to porous particles containing an iron-manganese bimetallic oxide portion impregnated with a sodium promoter. The iron is of iron oxide, Fe₃O₄, and the manganese addition can stabilize the iron oxide phase and form some FeMn spinel in which both manganese addition and sodium addition enhance the Fe₃O₄ crystallinity.

FIG. 3. Graph showing surface area determination for FeMn@Na.

### DETAILED DESCRIPTION

In one aspect, the present disclosure is directed to a porous catalyst useful in the conversion of carbon dioxide to one or more hydrocarbons. The porous catalyst contains at least (or exclusively) the following components: (i) a bimetallic oxide portion containing at least one of iron oxide and nickel oxide or carbide in combination with at least one oxide, hydroxide, and/or carbide of at least one of manganese (Mn), cobalt (Co), copper (Cu), yttrium (Y), zirconium (Zr), niobium (Nb), hafnium (Hf), zinc (Zn), and lanthanides, or a sub-combination of any of these; and (ii) an alkali metal oxide, hydroxide, or carbonate portion in contact with the bimetallic oxide portion; wherein the porous catalyst contains pores in the bimetallic oxide portion. Component (ii) may also be referred to as a promoter for component (i). Component (ii) may also be considered to be impregnated with, coated with, encapsulated with, or decorated with component (i), all of which may be within the scope of component (i) being in contact with component (ii). The porous catalyst composition may also be denoted as [component (i) metals] @ [component (ii)], such as FeMn@Na, FeCu@Na, FeZr@Na, FeMn@Li, FeMn@K, and so on. Notably, the term “oxide,” as used herein, is meant to include metal oxide compositions that include anions in addition to the oxide anion (O²⁻), and thus, the oxide compositions may include such compositions as oxide-hydroxides, oxide-halides, oxide-carbonates, oxide-sulfates, oxide-hydroxide-halides, oxide-hydroxide-carbonates, and oxide-carbonate-halides. However, in some embodiments, the oxide may exclude any of the above types of oxide compositions, or the oxide may exclusively contain the oxide anion or exclusively contain oxide and hydroxide anions.

In some embodiments, component (i), which is the bimetallic oxide portion, contains an iron oxide in combination with at least one oxide, hydroxide, and/or carbide of at least one of (or two or more of) Mn, Co, Cu, Y, Zr, Nb, Hf, Zn, and lanthanides, or a sub-combination of any of these (e.g., an iron oxide or carbide in combination with at least one oxide, hydroxide, and/or carbide of at least one of, or two or more of, Cu, Co, Mn, and Zr). In other embodiments, component (i) contains a nickel oxide in combination with at least one oxide, hydroxide, and/or carbide of at least one of (or two or more of) Mn, Co, Cu, Y, Zr, Nb, Hf, Zn, and lanthanides, or a sub-combination of any of these (e.g., a nickel oxide in combination with at least one oxide, hydroxide, and/or carbide of at least one of, or two or more of, Cu, Co, Mn, and Zr). In other embodiments, component (i) contains an iron-nickel oxide mixture in combination with at least one oxide, hydroxide, and/or carbide of at least one of (or two or more of) Mn, Co, Cu, Y, Zr, Nb, Hf, Zn, and lanthanides, or a sub-combination of any of these (e.g., an iron-nickel oxide mixture in combination with at least one oxide, hydroxide, and/or carbide of at least one of, or two or more of, Cu, Co, Mn, and Zr).

The iron oxide or carbide can be any of the iron oxides or carbides or iron oxide-hydroxides known in the art, including oxides of divalent and trivalent iron or a mixture thereof. The iron oxide may be, for example, any of the alpha, beta, gamma, or epsilon phases of Fe₂O₃, or FeO, or Fe₃O₄, or any of the iron oxide-hydroxides (e.g., goethite, akageneite, lepidocrocite, feroxyhyte, ferrihydrite, or green rust), or a combination of any two or more of these. The nickel oxide is typically NiO or a nickel oxide-hydroxide (e.g., NiO(OH)). Oxides of manganese include MnO, Mn₃O₄, Mn₂O₃, and MnO₂. Oxides of cobalt include CoO and Co₂O₃. Oxides of copper include Cu₂O and CuO. The term “lanthanide” refers to those elements having an atomic number of 57-71. The lanthanide elements are listed as follows: lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), and lutetium (Lu). The lanthanide elements are typically in a trivalent state in their oxide, hydroxide, and carbide compositions. Any one or a combination of the lanthanide elements may be included or excluded. In some embodiments, any one or more of the above elements is excluded from the porous catalyst, provided that the bimetallic portion contains at least one of iron oxide and nickel oxide in combination with at least one oxide, hydroxide, and/or carbide of at least one of manganese, cobalt, copper, yttrium, zirconium, niobium, hafnium, zinc, and lanthanides.

As noted above, component (ii) contains an alkali metal oxide, hydroxide, or carbonate portion in contact with the bimetallic oxide portion. Component (ii) functions to absorb the carbon dioxide and transport it into component (i), wherein component (i) is the reactive portion that converts the carbon dioxide to hydrocarbons. The alkali metal is necessarily in a positive valence state and can be, for example, lithium (Li), sodium (Na), potassium (K), rubidium (Rb), or a combination of two or more of any of these. In some embodiments, component (ii) includes (or is exclusively composed of) an alkali metal oxide. Some examples of alkali metal oxides include lithium oxide, sodium oxide, and potassium oxide. In other embodiments, component (ii) includes (or is exclusively composed of) an alkali metal hydroxide. Some examples of alkali metal hydroxides include lithium hydroxide, sodium hydroxide, and potassium hydroxide. In other embodiments, component (ii) includes (or is exclusively composed of) an alkali metal carbonate. Some examples of alkali metal carbonates include lithium carbonate, sodium carbonate, and potassium carbonate. In other embodiments, component (ii) includes (or is exclusively composed of) a combination of any two or all three of alkali metal oxide, alkali metal hydroxide, and alkali metal carbonate, such as a combination of alkali metal oxide and hydroxide, alkali metal oxide and carbonate, alkali metal hydroxide and carbonate, or alkali metal oxide, hydroxide, and carbonate.

Any of the exemplary embodiments for component (i) provided above can be combined with any of the exemplary embodiments for component (ii) provided above. In a first particular embodiment, the bimetallic oxide portion contains at least one of iron oxide and nickel oxide in combination with at least one oxide, hydroxide, and/or carbide (or at least one oxide and/or hydroxide) of at least one of copper, cobalt, manganese, and zirconium. In a second particular embodiment, the bimetallic oxide portion contains iron oxide in combination with at least one oxide, hydroxide, and/or carbide (or at least one oxide and/or hydroxide) of at least one of manganese, cobalt, copper, yttrium, zirconium, niobium, hafnium, zinc, and lanthanides. In a third particular embodiment, the bimetallic oxide portion contains iron oxide in combination with at least one oxide, hydroxide, and/or carbide (or at least one oxide and/or hydroxide) of at least one of copper, cobalt, manganese, and zirconium. In a fourth particular embodiment, the bimetallic oxide portion contains nickel oxide in combination with at least one oxide, hydroxide, and/or carbide (or at least one oxide and/or hydroxide) of at least one of copper, cobalt, manganese, and zirconium.

The porous catalyst described above contains pores in component (i), the bimetallic oxide portion. The pores permit the carbon dioxide to enter the catalyst and be converted into hydrocarbons. The pores are typically microporous, which corresponds to a pore size within a range of 0.1-2.5 nm or 0.5-2.5 nm, or more particularly, 0.1-2 nm, 0.2-2 nm, 0.5-2 nm, 0.1-1.5 nm, 0.2-1.5 nm, 0.5-1.5 nm, or 0.1-1 nm. In some embodiments, all (i.e., 100%) of the pores have a pore size within any of the foregoing pore size ranges. In other embodiments, at least or greater than 70%, 80%, 90%, or 95% of the pores have a pore size within any of the foregoing pore size ranges. Stated differently, 70%, 80%, 90%, or 95% (or range therein) of the total pore volume may be attributed to a pore size within any of the foregoing pore size ranges.

The porous catalyst can have a number of possible arrangements of components (i) and (ii) provided that components (i) and (ii) are in contact with each other and component (i) is covered or surrounded by component (ii), with component (ii) being uncoated and in free contact with its gaseous environment. Generally, components (i) and (ii) are not intermixed or intermingled, i.e., each component is separate and occupies distinct regions while in contact with each other.

A first exemplary arrangement of the porous catalyst is a core-shell particle arrangement, with the core being component (i) and the shell being component (ii). The shell is constructed of a layer of particles of component (ii) which are preferably tightly or closed packed. The core-shell particles generally have a spherical or approximately spherical or globular shape. Since the shell is composed of particles that are not completely flush with each other, interstitial voids (i.e., pores) are present in the shell, which permits the passage of gas through the shell and into the porous core. The shell at least partially encapsulates (or preferably, completely encapsulates) the core. Typically, the core is not constructed of an agglomeration of particles, but is rather a single porous mass. In each core-shell particle, component (ii) is uncoated and in free contact with its gaseous environment. The core-shell particles typically have a size in a range of 50-200 nm, which may or may not include particle sizes outside of the stated range. In various embodiments, the core-shell particles have a size of precisely or about, for example, 50, 60, 70, 80, 90, 100, 110, 120, 130, 140, 150, 160, 170, 180, 190, or 200 nm, or a particle size within a range bounded by any two of the foregoing values, e.g., 50-200 nm, 50-180 nm, 50-150 nm, 50-120 nm, 50-100 nm, 50-80 nm, 80-200 nm, 80-180 nm, 80-150 nm, 80-120 nm, or 80-100 nm. In some embodiments, all (i.e., 100%) of the particles have a particle size within any of the foregoing ranges. In other embodiments, at least or greater than 50%, 60%, 70%, 80%, 90%, or 95% of the particles have a particle size within any of the foregoing ranges.

The particles of component (ii) in the shell are generally smaller (e.g., no more than 50%, 25%, 10%, 5%, 2% or 1%) than the size of the core. In typical embodiments, particles of component (ii) may have any of the sizes provided above for the core-shell particle provided that the size of particles of component (ii) is smaller than the size of the core. The core may have any of the sizes provided above, or more typically, a size of 50-180 nm, 50-150 nm, or 50-100 nm. In some embodiments, the particles of component (ii) may be, for example, 1, 2, 5, 10, or 20 nm, or a particle size within a range bounded by any two of the foregoing values, e.g., 1-20 nm, 1-10 nm, 1-5 nm, or 1-3 nm.

A second exemplary arrangement of the porous catalyst is a layered arrangement in which a first layer containing component (ii) is in contact with a second layer containing component (i), wherein component (ii) is uncoated and in free contact with its gaseous environment. Component (i) is typically coated onto or bound to an inert substrate or support. In typical embodiments, a layer of particles of component (i) is disposed on a substrate or support and a layer of particles of component (ii) is disposed on the layer of component (i). The particles of components (i) and (ii) may independently have any of the particle sizes or ranges thereof as provided above. The particles of component (i) are porous as described above, with pore sizes as described above. Interstitial spaces may also be present in the layer of component (i), and these spaces can also function as pores. As earlier mentioned, the layer of component (ii) is also porous, typically due to interstitial spaces between the particles.

The porous catalyst described above can be produced by methods well known in the art. In typical embodiments, bimetallic catalysts with varying atomic ratios can be prepared via a templating method using the oxalate route. A solution containing an appropriate amount of metal nitrates (example Co(NO₃)·6H₂0) or metal sulphate (FeSO₄·7H₂O) may be dissolved in high purity water at 35° C. A solution of 1.5 mol excess of oxalic acid (for example, 0.3 mol oxalic acid to 250 mL of water) to that of the metal salts, may also be dissolved in high purity water at 35° C. The metal-containing solution may be added dropwise to the oxalic acid solution while stirring, and stirring can be continued at a suitable temperature, such as 35° C., for a suitable time period, such as 1 hour, after complete addition. The heat can be removed and stirring continued until room temperature is reached. The precipitated complexes may be filtered and washed, typically a minimum of three times with deionized water, and the solid may be dried under vacuum at 80-100° C. for 24 hours. The alkali metal (such as potassium nitrate) may be added at varying atomic ratios, i.e., K: (Fe+Co) 0.001, 0.002, 0.003, 0.004, 0.005, 0.01, 0.02, 0.03, 0.04, 0.05 by the incipient wetness technique, wherein the alkali salt may be dissolved in a minimum of water and added to the metal oxide support followed by slow evaporation of solvent whereby then the catalyst can be dried at 100° C. in a vacuum oven.

In another aspect, the present disclosure is directed to a method of converting carbon dioxide (CO₂) to one or more hydrocarbons, at least a portion of which contain at least four carbon atoms. In the method, carbon dioxide and hydrogen (H₂) gases (typically a mixture thereof in an input gas stream, but may be inputted separately) are contacted with the above-described porous catalyst, which include any of the porous catalyst compositions and arrangements described above, at a temperature of 100-800° C. and a pressure of 1-20 atm that together result in conversion of the carbon dioxide to the one or more hydrocarbons.

The temperature employed in the process (i.e., the temperature of the gas stream containing CO₂ and/or the temperature of the catalyst) may be precisely or about, for example, 100, 150, 200, 250, 300, 350, 400, 500, 600, 700, or 800° C., or a temperature within a range bounded by any two of the foregoing values, e.g., 100-800° C., 200-800° C., 250-800° C., 270-800° C., 300-800° C., 350-800° C., 375-800° C., 400-800° C., 450-800° C., 100-500° C., 200-500° C., 250-500° C., 270-500° C., 300-500° C., 350-500° C., 375-500° C., 400-500° C., 450-500° C., 100-400° C., 200-400° C., 250-400° C., 270-400° C., 300-400° C., 350-400° C., 100-350° C., 200-350° C., 250-350° C., or 300-350° C. The input gas stream may or may not be heated before contacting the catalyst. If heated, the input gas stream may be heated to any of the temperatures provided above.

The pressure employed in the process (i.e., the pressure of the input gas stream containing the CO₂ and H₂ gases) may be ambient pressure (about 1 atm) or an elevated pressure above 1 atm when contacting the porous catalyst. When an elevated pressure is used, the pressure may be precisely, at least, or above, for example, 2, 5, 8, 10, 15, 18, or 20 atm, or a pressure within a range bounded by any two of the foregoing values (e.g., 1-20 atm, 1-18 atm, 1-15 atm, 1-10 atm, 1-8 atm, 1-5 atm, 2-20 atm, 2-10 atm, 2-5 atm, 5-20 atm, 5-15 atm, 5-10 atm, 10-20 atm, 10-15 atm, or 15-20 atm).

Any of the temperatures (or ranges thereof) provided above may be combined with any of the pressures (or ranges thereof) provided above. Notably, lower temperatures may employ higher pressures to achieve the same or similar result in the hydrocarbon product. For example, a temperature of at least 300 or 400° C. may achieve a desirable hydrocarbon product distribution and yield at ambient pressure or low pressure (e.g., 2-5 atm) while a temperature of 100-200° C. may be used in combination with a pressure of 5-20 atm to achieve a similarly desirable hydrocarbon product distribution and yield.

The CO₂ and H₂ gases are typically present in the input gas stream in a CO₂:H₂ molar (or volume) ratio of 0.1 to 1 (i.e., 0.1:1 to 1:1, or equivalently, 1:10 to 1:1). In different embodiments, the input gas stream includes CO₂ and H₂ gases in a CO₂:H₂ molar (or volume) ratio of 1:10, 1:9, 1:8, 1:7, 1:6, 1:5, 1:4, 1:3, 1:2, or 1:1, or a ratio within a range bounded by any two of the foregoing ratios (e.g., 1:10-1:1, 1:5-1:1, 1:4-1:1, or 1:3-1:1).

The input gas stream makes contact with the porous catalyst for any suitable gas-phase residence time at any of the gas ratios, pressures, or temperatures provided above. The residence time is typically within a range of 1 second to 24 hours, depending on the conditions employed. In some embodiments, the residence time may be longer, e.g., 30, 35, or 40 hours. In different embodiments, and depending on the conditions used, the residence time may be 1 second, 5 seconds, 10 seconds, 30 seconds, 1 minute, 5 minutes, 10 minutes, 30 minutes, 1 hour, 2 hours, 3 hours, 4 hours, 5 hours, 6 hours, 12 hours, 18 hours, or 24 hours, or a residence time within a range bounded by any two of the foregoing values (e.g., 1 second to 24 hours, 1 second to 12 hours, 1 second to 6 hours, 1 second to 2 hours, 1 second to 1 hour, 1 second to 30 minutes, 1 second to 10 minutes, 1 second to 1 minute, 1-30 seconds, 1 minute to 24 hours, 1 minute to 12 hours, 1 minute to 6 hours, 1-40 hours, 1-30 hours, 6-40 hours, 6-30 hours, 6-24 hours, 10-40 hours, 10-30 hours, or 10-24 hours).

The method employs any suitable weight hourly space velocity (WHSV) of CO₂, wherein it is known that the WHSV is at least in part determined by the feed content and gas-phase residence time. The WHSV is typically in a range of 0.2-3 h⁻¹. In different embodiments, and dependent on the feed content, residence time, and other factors, the WHSV may be precisely or about, for example, 0.2 h⁻¹, 0.3 h⁻¹, 0.4 h⁻¹, 0.5 h⁻¹, 0.6 h⁻¹, 0.7 h⁻¹, 0.8 h⁻¹, 0.9 h⁻¹, 1 h⁻¹, 1.2 h⁻¹, 1.4 h⁻¹, 1.6 h⁻¹, 1.8 h⁻¹, 2 h⁻¹, 2.2 h⁻¹, 2.4 h⁻¹, 2.6 h⁻¹, 2.8 h⁻¹, or 3 h⁻¹, or a WHSV within a range bound by any two of the foregoing values (e.g., 0.2-3 h⁻¹, 0.2-2.5 h⁻¹, 0.2-2 h⁻¹, 0.3-3 h⁻¹, 0.3-2.5 h⁻¹, 0.3-2 h⁻¹, 0.4-3 h⁻¹, 0.4-2.5 h⁻¹, 0.4-2 h⁻¹, 0.5-3 h⁻¹, 0.5-2.5 h⁻¹, 0.5-2 h⁻¹, 1-3 h⁻¹, 1-2.5 h⁻¹, or 1-2 h⁻¹).

When contacting the input gas stream, the porous catalyst may be housed in any suitable reactor design, such as a packed-bed reactor, pellet-bed reactor, trickle bed, bubble bed, stirred tank or a fluidized bed reactor. In some embodiments, the reactor has a 3D printed (e.g., into microchannels) or electrospun reactor design to better overcome pressure drop issues and improve the product distribution. Coating of the catalyst onto monoliths may also be advantageous in overcoming pressure drop issues. Electrospun reactors (e.g., containing electrospun polymetallic fibers) can also improve the metal distribution, which can improve product selectivity. In some embodiments, the process excludes a static autoclave batch reactor, packed-bed, fixed bed, pellet-bed, trickle bed, bubble column or stirred tank or fluidized bed reactor.

The carbon dioxide being converted may be produced by any known source of carbon dioxide. The source of carbon dioxide may be, for example, ambient air, a combustion source (e.g., from burning of fossil fuels in an engine or generator), natural gas emission (natural gas combined cycle), commercial biomass fermenter (e.g., ethanol fermentation), flue gas, or commercial carbon dioxide-methane separation process for gas wells.

The process described above is particularly suited for producing hydrocarbons (particularly olefins) containing at least four carbon atoms. Typically, at least 20 vol % of the hydrocarbons produced by the process contain at least four carbon atoms. In various embodiments, at least or greater than 20, 30, 40, 50, 60, 70, 80, 85, 90, or 95 vol % of the hydrocarbons (or more particularly, olefins) produced by the method contain at least four carbon atoms. In further or separate embodiments, at least or greater than 1, 2, 5, 10, 15, 20, 30, 40, 50, 60, 70, or 80 vol % of the hydrocarbons (or more particularly, olefins) produced by the method contain at least five carbon atoms. In further or separate embodiments, at least or greater than 1, 2, 5, 10, 15, 20, 30, 40, or 50 vol % of the hydrocarbons (or more particularly, olefins) produced by the method contain at least six, seven, eight or a higher number of carbon atoms. In some embodiments, the process produces one or more hydrocarbons (or more particularly, olefins) containing up to 8, 10, 12, 15, 18, or 20 carbon atoms. Any of the minimum and maximum number of carbon atoms disclosed above may be combined to result in a range of carbon atoms (e.g., 4-20, 4-15, or 4-13 carbon atoms).

In some embodiments, hydrocarbons (or more particularly, olefins) containing less than four carbon atoms are not produced or are produced in a minor amount, typically no more than or less than 20, 15, 10, 5, 2, or 1 vol %. In some embodiments, hydrocarbons (or more particularly, olefins) containing precisely or less than three carbon atoms are not produced or are produced in a minor amount, typically no more than or less than 20, 15, 10, 5, 2, or 1 vol %. In some embodiments, hydrocarbons containing two carbon atoms (e.g., ethylene and/or ethane) are not produced or are produced in a minor amount, typically no more than or less than 20, 15, 10, 5, 2, or 1 vol %. In some embodiments, methane is not produced or is produced in a minor amount, typically no more than or less than 10, 5, 2, or 1 vol %. In some embodiments, aromatic molecules are not produced or are produced in a minor amount, typically no more than or less than 10, 5, 2, or 1 vol %. In some embodiments, oxygen-containing molecules are not produced or are produced in a minor amount, typically no more than or less than 10, 5, 2, or 1 vol %. In some embodiments, carbon monoxide (CO) is not produced or is produced in a minor amount, typically no more than or less than 10, 5, 2, or 1 vol %. In some embodiments, alkanes (i.e., paraffins) are not produced or are produced in a minor amount, typically no more than or less than 50, 40, 30, 20, 10, 5, 2, or 1 vol %.

In some embodiments, at least a portion of the hydrocarbons produced by the method (which may be hydrocarbons containing above and below four carbon atoms) are olefins. For example, in some embodiments, at least or greater than 20, 30, 40, 50, 60, 70, 80, or 90 vol % (or 100 vol %) of the hydrocarbons produced by the method (i.e., hydrocarbons containing above and below four carbon atoms) are olefins. In some embodiments, at least a portion of the hydrocarbons containing at least or greater than four, five, and/or six carbon atoms, as may be produced by the method, are olefins. For example, in some embodiments, at least or greater than 20, 30, 40, 50, 60, 70, 80, or 90 vol % (or 100 vol %) of the hydrocarbons containing at least or greater than four, five, or six carbon atoms, as may produced by the method, are olefins. Moreover, any of the above embodiments may be combined to provide a more tailored product distribution.

Examples have been set forth below for the purpose of illustration and to describe certain specific embodiments of the invention. However, the scope of this invention is not to be in any way limited by the examples set forth herein.

### Examples

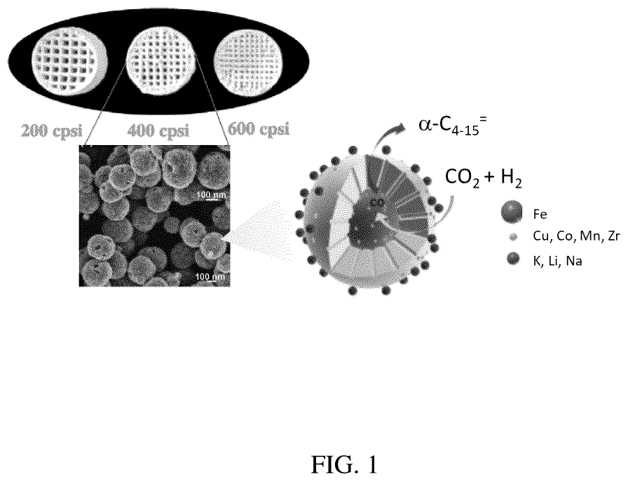

Surface area and porosity of particles of porous FeMn@Na were determined by nitrogen gas physisorption, wherein FeMn@Na corresponds to porous particles containing an iron-manganese bimetallic oxide portion impregnated with a sodium promoter. The iron is an iron oxide, Fe₃O₄, and the manganese addition can stabilize the iron oxide phase and form some FeMn spinel in which both manganese addition and sodium addition enhance the Fe₃O₄ crystallinity. The particles have the arrangement shown in FIG. 1. Density was tested using a pycnometer. XPS was used to analyze the surface of the samples and their oxidation states. High resolution TEM (HRTEM) and EDX provided particle size and elemental surface distribution. Temperature-programmed profiles provided analysis of oxygen, acidic or basic sites. Inductively coupled plasma-optical emission spectroscopy (ICP-OES) was used to characterize the elemental composition. X-ray diffraction (XRD) was used to elucidate the crystalline structures.

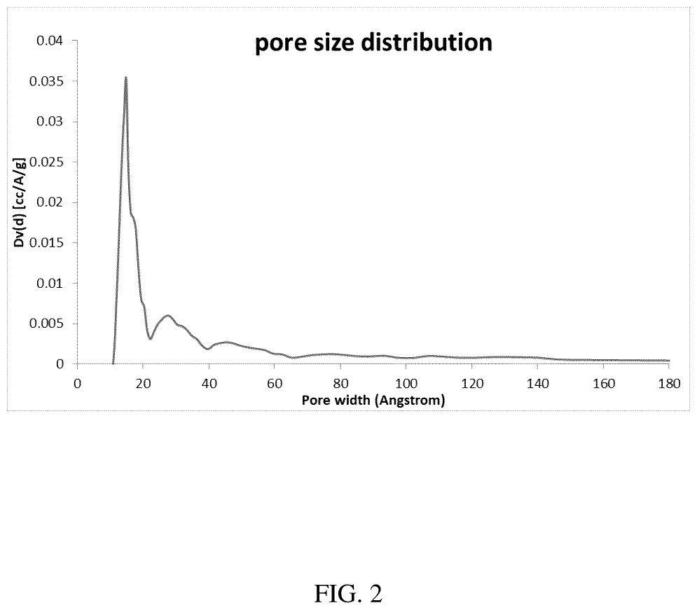

FIG. 2 is a graph showing the pore size distribution of porous FeMn@Na particles, as determined by nitrogen gas physisorption. As shown, the pore size of a typical particle is between 1-2 nm where most of the pore volume resides. There also appears to be some pore volume above micropores at 2-4 nm and a small amount of volume from pore sizes at 4-6 nm.

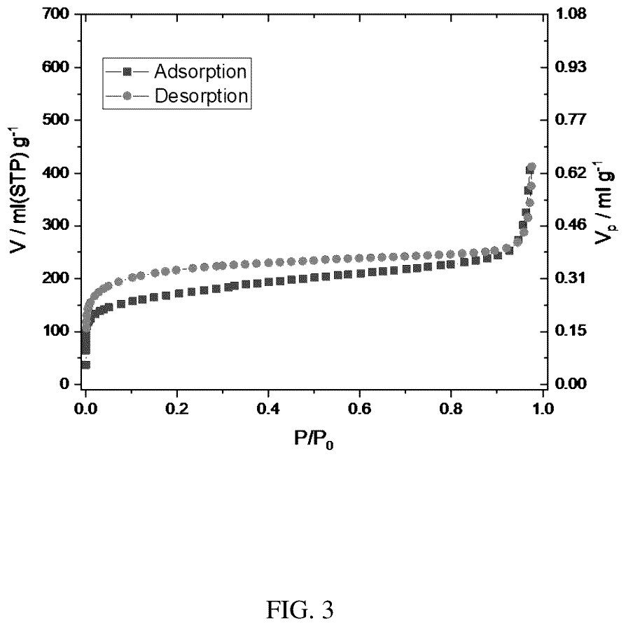

FIG. 3 is a graphical plot of the surface area for the porous FeMn@Na particles. As shown, the surface area is approximately 570 m²/g.

While there have been shown and described what are at present considered the preferred embodiments of the invention, those skilled in the art may make various changes and modifications which remain within the scope of the invention defined by the appended claims.

What is claimed is:

1. A porous catalyst useful in the conversion of carbon dioxide to one or more hydrocarbons, the porous catalyst comprising:
(i) a bimetallic oxide portion comprising at least one of iron oxide and nickel oxide in combination with at least one oxide, hydroxide, and/or carbide of at least one of manganese, cobalt, copper, yttrium, zirconium, niobium, hafnium, zinc, and lanthanides; and
(ii) an alkali metal oxide, hydroxide, or carbonate portion in contact with the bimetallic oxide portion;
wherein the porous catalyst contains pores in the bimetallic oxide portion.

2. The porous catalyst of claim 1, wherein at least 90 vol % of the pores have a size within a range of 0.5-2.5 nm.

3. The porous catalyst of claim 1, wherein the bimetallic oxide portion comprises at least one of iron oxide and nickel oxide in combination with at least one oxide, hydroxide, and/or carbide of at least one of copper, cobalt, manganese, and zirconium.

4. The porous catalyst of claim 1, wherein the bimetallic oxide portion comprises iron oxide in combination with at least one oxide, hydroxide, and/or carbide of at least one of manganese, cobalt, copper, yttrium, zirconium, niobium, hafnium, zinc, and lanthanides.

5. The porous catalyst of claim 1, wherein the bimetallic oxide portion comprises iron oxide in combination with at least one oxide, hydroxide, and/or carbide of at least one of copper, cobalt, manganese, and zirconium.

6. The porous catalyst of claim 1, wherein the porous catalyst is comprised of particles having a core-shell arrangement in which the core comprises said bimetallic oxide portion and the shell comprises said alkali metal oxide, hydroxide, or carbonate portion.

7. The porous catalyst of claim 6, wherein at least 90% of the particles have a particle size in a range of 50-200 nm.

8. The porous catalyst of claim 1, wherein the porous catalyst comprises a first layer containing said bimetallic oxide portion and a second layer containing said alkali metal oxide, hydroxide, or carbonate portion, wherein said second layer is in contact with said first layer, and said second layer is uncoated and in free contact with its gaseous environment.

9. A method of converting carbon dioxide to one or more hydrocarbons at least a portion of which contain four carbon atoms, the method comprising contacting carbon dioxide (CO₂) and hydrogen (H₂) gases with a porous catalyst at a temperature of 100-800° C. at a pressure of 1-20 atm to result in conversion of the carbon dioxide to said one or more hydrocarbons, wherein the porous catalyst comprises:
(i) a bimetallic oxide portion comprising at least one of iron oxide and nickel oxide in combination with at least one oxide, hydroxide, and/or carbide of at least one of manganese, cobalt, copper, yttrium, zirconium, niobium, hafnium, zinc, and lanthanides; and
(ii) an alkali metal oxide, hydroxide, or carbonate portion in contact with the bimetallic oxide portion;
wherein the porous catalyst contains pores in the bimetallic oxide portion.

10. The method of claim 9, wherein at least 50 vol % of said hydrocarbons contain at least four carbon atoms.

11. The method of claim 9, wherein at least 60 vol % of said hydrocarbons contain at least four carbon atoms.

12. The method of claim 9, wherein at least 70 vol % of said hydrocarbons contain at least four carbon atoms.

13. The method of claim 9, wherein at least 80 vol % of said hydrocarbons contain at least four carbon atoms.

14. The method of claim 9, wherein at least a portion of said hydrocarbons are olefins containing at least four carbon atoms.

15. The method of claim 9, wherein at least 50 vol % of said hydrocarbons are olefins containing at least four carbon atoms.

16. The method of claim 9, wherein the bimetallic oxide portion of the porous catalyst comprises at least one of iron oxide and nickel oxide in combination with at least one oxide, hydroxide, and/or carbide of at least one of copper, cobalt, manganese, and zirconium.

17. The method of claim 9, wherein the bimetallic oxide portion of the porous catalyst comprises iron oxide in combination with at least one oxide, hydroxide, and/or carbide of at least one of manganese, cobalt, copper, yttrium, zirconium, niobium, hafnium, zinc, and lanthanides.

18. The method of claim 9, wherein the bimetallic oxide portion of the porous catalyst comprises iron oxide in combination with at least one oxide, hydroxide, and/or carbide of at least one of copper, cobalt, manganese, and zirconium.

19. The method of claim 9, wherein the porous catalyst is comprised of particles having a core-shell arrangement in which the core comprises said bimetallic oxide portion and the shell comprises said alkali metal oxide, hydroxide, or carbide portion.

20. The method of claim 9, wherein the porous catalyst comprises a first layer containing said bimetallic oxide portion and a second layer containing said alkali metal oxide, hydroxide, or carbonate portion, wherein said second layer is in contact with said first layer, and said second layer is uncoated and in free contact with its gaseous environment.
